(19) United States
(12) Patent Application Publication
(10) Pub. No.: US 2013/0299382 A1
(43) Pub. Date: Nov. 14, 2013

(54) Germ Barrier Travel Kit

(71) Applicant: Patricia Ann Mullis, Campobello, SC (US)

(72) Inventor: Patricia Ann Mullis, Campobello, SC (US)

(21) Appl. No.: 13/471,049

(22) Filed: May 14, 2012

Related U.S. Application Data
(60) Provisional application No. 61/561,310.

(51) Int. Cl. A61B 19/02

(52) U.S. Cl. 206/570

(57) ABSTRACT

A germ/barrier travel kit sized, shaped and configured to carry articles necessary for germ prevention and sterilization. In one embodiment, the germ/barrier travel kit consists of a bag/container including but not limited to: toilet seat cover, face mask, gloves, head rest cover and hygiene/disinfecting wipes. However, the germ/barrier travel kit may also be configured to carry additional wipes or barriers. The germ/barrier travel kit is configured to permit ready and easy access to articles needed by travelers to defend against germs and preventing disease transmission.

The application claims priority to U.S. provisional patent application No. 61561310 The present disclosure relates to a kit and more particularly to an application-specific kit and related method.

### BACKGROUND AND SUMMARY

1. Field of Invention

The present invention relates to a portable germ/barrier travel kit. More specifically, a kit that consists of multiple disposable germ/barrier, sanitation, and disinfectant items carried in a bag/container.

2. Background of Invention

This section provides background information related to the present disclosures which is not necessarily prior art. This section also provides a general summary of the disclosure, and is not a comprehensive disclosure of its full scope or all of its features.

The World Health Organization estimates that each year millions of people die from bacteria related diseases alone. Recently, it was reported that airplanes are some of the dirtiest places. Bacteria particles were found on meal trays as well as numerous other places on the airplanes. This is just one example of bacterial agents present in our daily lives. Daily people travel either by plane, bus, train or automobile.

The following listed patents further illustrate less relevant kits: Wound care kit (U.S. Pat. No. 7,857,137 patented December 2010 by Law/Hess) contains wound coverings as well as wound care treatments. It contains only articles pertaining to wound care.

First aid kits (U.S. Pat. Nos. 1,436,406; 2,135,238 patented November 1922 and 1938 by Schulz; Malik) are typically a flexible resealable bag or other type of container. The kits are used to treat minor injuries. Pocket first aid kits (U.S. Pat. Nos. 2,276,766; 2,377,117 patented March 1942 and May 1945 De Witt; Watkins) may be used in virtually any household or business and the kits often include bandages of varying sizes and shapes. These kits often include products that provide individuals the items necessary to address injuries.

Because conventional kits are generic and may be used in virtually any household or business, conventional kits often include bandages of varying size and shape to accommodate a wide range of applications. Likewise, such kits often include a myriad of medications and other products in an effort to provide the kit with the ability to address a wide range of injuries and/or medical conditions. Conventional kits are not application specific and, as a result are typically cumbersome and often times include a large container to accommodate a wide range of products designed to address numerous injuries and/or medical conditions.

### SUMMARY OF INVENTION

A germ/barrier travel kit is application specific for the prevention of germ/disease transmission encountered with ordinary travels. The germ/barrier travel kit provides the traveler with a portable compact kit that may be carried with them on travels and contains disposable items necessary for the minimization in the disease transmission probability. Examples of these items include but not limited to toilet seat covers, face masks, gloves, head rest covers and germ defense/prevention wipes. This kit preferably permits reuse thereof.

Further below you will find specific examples intended for the purposes of illustration and are not intended to limit the scope of the present disclosure. Other items can and will be added as they become available or are invented will be included in for mentioned kit.

### BRIEF DESCRIPTION OF DRAWINGS

FIG. 1

1 resealable bag/container which is flexible and relatively small and air tight

2 shows the bag/container with contents in 4, 5, 6, 7, 8, 9

3 hole in bag/container a hanger opening for detachably securing said bag/container to a hanger for display or stand

FIG. 2

4 Packaged toilet seat cover suitable in size; individually sealed for germ barrier protection

5 Packaged face mask suitable in size; individually sealed for germ barrier protection

6 Packaged gloves suitable in size; individually sealed for germ barrier protection

7 Packaged head rest seat cover suitable for any transportation; individually sealed for germ barrier protection

8 Packaged germicidal towelettes suitable in size; individually sealed for personal hygiene

9 Packaged disinfectant towelettes suitable in size; individually sealed for surface cleaning

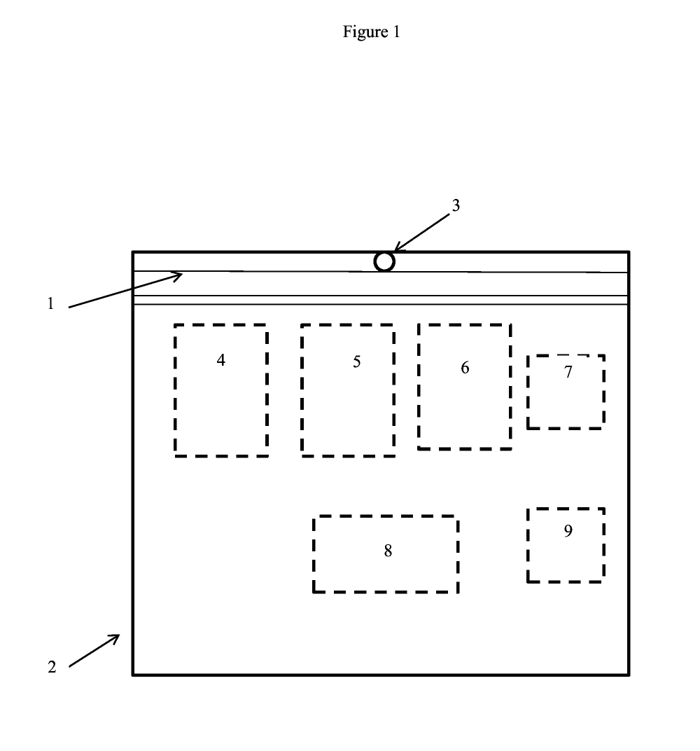

FIG. 1:

Sanitary Kit Container (Plan View)
1. Container is made of water tight material. Shape and volume may vary based on amount of amount of contents. Standard volume is approximately one quart, but may range from four ounces to one gallon.
2. Container opening is resealable plastic strips, zipper, or other resealable mechanism.
3. Hole for hanging kit. (optional)

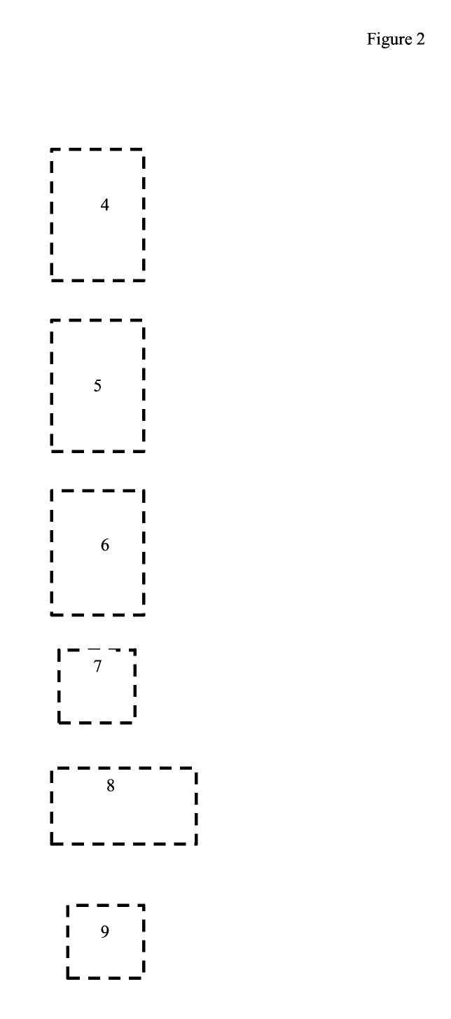

FIG. 2:

Sanitary Kit Contents.

The contents of the sanitary kit include the following items in varying quantities as needed. Some items may be omitted in smaller kits.
4. Individually wrapped toilet seat cover. Plastic, paper, or other germ barrier material shaped to lay over standard toilet seats.
5. Individually wrapped face mask. Breathable mask to cover the nose and mouth that straps behind the head.
6. Individually wrapped disposable gloves. Rubber, latex, vynol, or other germ barrier material disposable gloves.
7. Individually wrapped headrest cover: Paper, plastic or other germ barrier material shaped to lay over a (car/plane/train/bus) seat headrest.
8. Individually wrapped alcohol or other disinfecting hand wipes.
9. Individually wrapped disinfecting wipes for non-skin surfaces.

I claim:

1. A germ/barrier travel kit comprising of an outer resealable bag/container, contents inside are selected for germ defensive and prevention. The contents of the kit will vary based on the intended application. The kit consists of a resealable bag/container which is flexible and relatively small such that it can be conveniently stored in a variety of locations and, in particular, in locations associated with the particular activity for which the kit is intended. The kit is compact and may easily be carried in luggage, purses or easily hidden. The bag/container contains a hanger opening for detachably securing said bag/container to a hanger for display or stand. Barrier protection components include toilet seat covers, face masks, gloves and head rest covers.
Germ fighting components include towelettes for wiping self and/or surfaces. A kit comprising: a flexible bag/container and numerous components in the bag, the components being selected from a group of components to make an application-specific kit, the group of components selected from the group consisting of germ barriers, germicides and germ disposable sanitary items.
1. The kit of claim 1, wherein said bag/container is fluid-tight.
2. The kit of claim 1, wherein said bag/container includes a closable opening.
3. The kit of claim 2, wherein said bag/container has a closable opening is resealable.
4. The kit of claim 1, wherein said bag/container is compact.
5. The kit of claim 1, wherein said bag/container further includes a hanger opening for detachably securing said bag/container to a hanger for display or stand.
6. The kit of claim 1, wherein said components in the bag/container include disposable items commonly used by travelers and may be carried on plane, train or other transportation.
7. The kit of claim 6, wherein said particular travel components consists of germ barriers and disinfecting items.
Packaged toilet seat cover suitable in size; individually sealed for germ barrier protection
Packaged face mask suitable in size; individually sealed for germ barrier protection
Packaged gloves suitable in size; individually sealed for germ barrier protection
Packaged head rest seat cover suitable for any transportation; individually sealed for germ barrier protection
Packaged germicidal towelettes suitable in size; individually sealed for personal hygiene
Packaged disinfectant towelettes suitable in size; individually sealed for surface cleaning
8. The kit of claim 1, wherein said bag/container may include trademark/logo.
